(19) United States
(12) Patent Application Publication
(10) Pub. No.: US 2016/0067432 A1
(43) Pub. Date: Mar. 10, 2016

(54) POSITIVE PRESSURE DEVICE

(71) Applicant: Peron Pierre, Brooklyn, NY (US)

(72) Inventor: Peron Pierre, Brooklyn, NY (US)

(21) Appl. No.: 14/942,578

(22) Filed: Nov. 16, 2015

Related U.S. Application Data
(63) Continuation-in-part of application No. 13/094,536, filed on Apr. 26, 2011, now abandoned.
(60) Provisional application No. 61/347,077, filed on May 21, 2010.

(51) Int. Cl. A61M 16/00; A61M 16/06; A61M 16/20; A61M 16/04; A61M 16/08

(52) U.S. Cl. CPC A61M 16/0057; A61M 16/0003; A61M 16/04; A61M 16/0816; A61M 16/208; A61M 16/0666; A61M 2016/003; A61M 2205/3334

(57) ABSTRACT

A positive pressure device used with an air source which provides transmission of continuous positive pressure into the lungs using an inspiratory line and a one-way valve associated with the inspiratory line. This one-way valve will not allow exhaled air to return through the inspiratory line. The pressure in the device may be measured with a monitor line which is connected to the air source and the disclosed positive pressure device. An adjustable positive end expiratory pressure valve connected to the expiratory line restricts flow of the exhaled air while permitting release of the exhaled air from an expiratory line to atmospheric pressure. The adjustable positive end expiratory pressure valve is configured to cooperate with the one-way valve to permit adjustment of an inflation pressure of the lungs of the patient.

### CROSS-REFERENCE TO RELATED APPLICATIONS AND CLAIMS OF PRIORITY

This application is a continuation-in-part of U.S. application Ser. No. 13/094,536, filed Apr. 26, 2011, which claims the benefit of U.S. Provisional Application No. 61/347,077, filed May 21, 2010. The complete disclosures of the applications are incorporated herein by reference and the benefit of priority of each application is claimed.

### FIELD OF THE INVENTION

The invention relates to the field of positive pressure breathing aide devices, including in exemplary embodiments a positive pressure (endo)tracheal device and a positive pressure nasal device, as well as methods of making and using the same, including in connection with individuals experiencing periods of apnea, hypoxia, atelectasis, and otherwise in need or benefit of positive pressure breathing assistance.

### BACKGROUND

There are a number of solutions for applying positive pressure for individuals with periods of apnea, hypoxia, atelectasis, increased work of breathing, and the like. Some of these solutions attempt to use conventional mechanics, but these solutions fail to meet the new requirements, which are the economical, environmental, physiological factors for today's rapidly changing medical environment. The positive pressure devices of exemplary embodiments disclosed herein may satisfy these requirements and to allow easy access and with comfortable, safe, and consistent operable connections.

### SUMMARY

A first aspect of the invention provides a positive pressure endotracheal device (PPETD) used with an air source which provides transmission of continuous positive pressure into the lungs using an inspiratory line and a one-way valve associated with (e.g., placed within) the inspiratory tube. This one-way valve will not allow pressure to return through the inspiratory line to the air source. The pressure is measured with a monitor line which is connected to the source and the positive pressure endotracheal device. As insufflations occur the lungs expand creating an increasing pressure gradient. This lung pressure is maintained due to the presence of a cuffed (inflated around the placed endotracheal tube) inserted tube. Involuntary exhalation phase occurs as follows: The lung exhalation pressure follows the path of least resistance, which is through a second line exhalation tube. A positive end expiratory pressure (PEEP) valve associated with (e.g., placed inside) the exhalation tube maintains lung expansion, at a predetermined adjustable pressure, while allowing the release of excess exhaled lung pressure.

It would be desirable to have a device which when applied will improve oxygenation and decrease the work of breathing while simultaneously maintaining positive end expiratory pressure (PEEP). Furthermore, it is desirable to have a device which is environmentally safe. The positive pressure endotracheal device of the first aspect of the present invention utilizes air coming from a source, i.e., wall O2, O2 tank, and/or an air concentrator. Still further this device is cost efficient and will meet the need in the medical industry to have a tool to improve the lives of many individuals with poor oxygenation and lung complications. This endotracheal device will inflate the lungs and allow passive exhalation to atmospheric pressure while maintaining a sustained positive end expiratory pressure with the use of a PEEP valve.

The first aspect of the present invention facilitates lung recruitment with the use of the one-way valve, placed proximal in the inspiratory line to create a pressure differential, allowing the one-way valve to remain open. A PEEP valve placed distal in the expiratory line creates an increased pressure gradient within the lungs, while allowing passive exhalation in a controlled manner.

An embodiment of the first aspect of the present invention includes: 1) a WYE endotracheal connector, 2) a pressure release port, 3) a pressure monitor port, 4) a dispenser port, 5) an inspiratory line, 6) a one-way valve, 7) a smooth walled expiratory line, and 8) a PEEP valve. The positive pressure endotracheal device entails a continuous transmission of positive pressure into the lungs of a spontaneously breathing patient using a cuffed endotracheal tube.

According to a second aspect of the invention, a method is provided which includes the following assembly and administration steps. A WYE endotracheal connector is used to interface with the trachea, using a cuffed endotracheal tube. A pressure release port placed on the proximal end of the molded WYE interface is used to facilitate emergency pressure release. A pressure monitor port is positioned vertically on the molded WYE endotracheal connector to monitor lung pressure. An internal one-way valve prevents pressure returning into the inspiratory line. A smooth walled exhalation line is connected to the expiratory side of the WYE connector and a PEEP valve connected on a distal end thereof provides resistance during the exhalation phase, allowing predetermined exhaust lung volume.

A third aspect of the invention provides a positive pressure nasal device, including an inspiratory line adapted to accept inspiratory air from a positive pressure air source at a positive air pressure, a one-way valve connected to the inspiratory line to permit one-way flow of the inspiratory air through the inspiratory line, a patient nasal interface connected to the inspiratory line and configured to receive the inspiratory air from the inspiratory line and conduct the inspiratory air to the lungs of the patient, an expiratory line connected to the patient nasal interface and configured to conduct air exhaled from the lungs of the patient, and an adjustable positive end expiratory pressure valve connected to the expiratory line for permitting restricted release of the exhaled air from the expiratory line into atmospheric pressure, the adjustable positive end expiratory pressure valve being configured to cooperate with the one-way valve to permit adjustment of an inflation pressure of the lungs of the patient to reach a continuous positive air pressure that exceeds the positive air pressure provided by the positive pressure air source.

Another aspect of the present invention involves a method of using a positive pressure device, involving delivering inspiratory air from a positive pressure air source through an inspiratory line to a patient wearing a patient nasal interface, the inspiratory line being associated with a one-way valve configured to permit one-way flow of the inspiratory air through the inspiratory line to the patient nasal interface, and controlling an adjustable positive end expiratory pressure valve connected to the patient nasal interface through an expiratory line to adjust an inflation pressure of the lungs of the patient to reach a continuous positive air pressure that exceeds the positive air pressure provided by the positive pressure air source.

Aspects of the present invention are unique in that they are structurally different from other known devices or solutions. For example, a one-way valve placed inside or outside a single inspiratory line transmits and maintains a positive pressure into the lungs and facilitates lung expansion and alveoli recruitment when used with a cuffed endotracheal/tracheal tube or patient nasal interface. A sustained recruitment is established. Exhaled lung volume is controlled with the use of a PEEP valve placed within or otherwise used in association with the expiratory line, which will maintain alveoli recruitment and facilitate a predetermined lung exhaust.

Other aspects and embodiments of the invention, including devices, components, assemblies, apparatus, kits, methods and processes of making and using, and the like which constitute part of the invention, will become more apparent upon reading the following detailed description of the exemplary embodiments.

### BRIEF DESCRIPTION OF THE DRAWINGS

The accompanying drawings are incorporated in and constitute a part of the specification. The drawings, together with the general description given above and the detailed description of the exemplary embodiments and methods given below, serve to explain principles of the invention. In such drawings:

FIG. 1 is a perspective view of a positive pressure device according to a first exemplary embodiment;

FIG. 2 is a flow diagram of an exemplary method associated with the first exemplary embodiment;

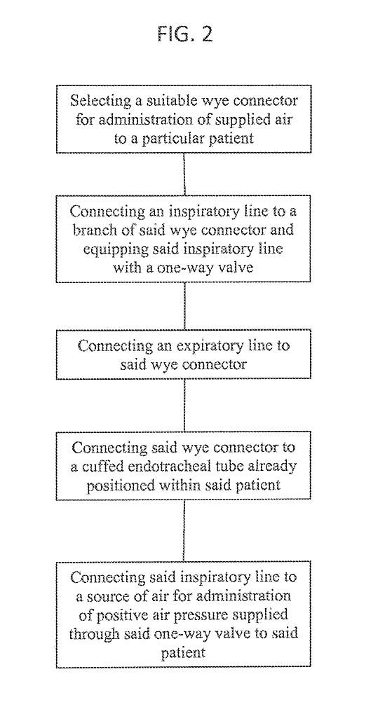

FIG. 3 is a perspective view of a positive pressure device according to a second exemplary embodiment; and

FIG. 4 is a flow diagram of an exemplary method associated with the second exemplary embodiment.

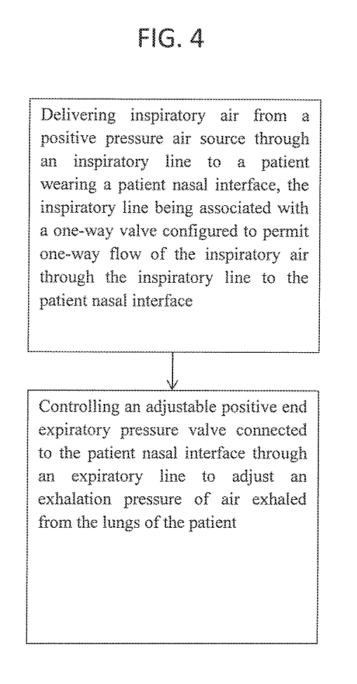

### DETAILED DESCRIPTION OF EXEMPLARY EMBODIMENTS AND METHODS

Reference will now be made in detail to the exemplary embodiments and methods as illustrated in the accompanying drawings, in which like reference characters designate like or corresponding parts throughout the drawings. It should be noted, however, that the invention in its broader aspects is not necessarily limited to the specific details, representative materials and methods, and illustrative examples shown and described in connection with the exemplary embodiments and methods.

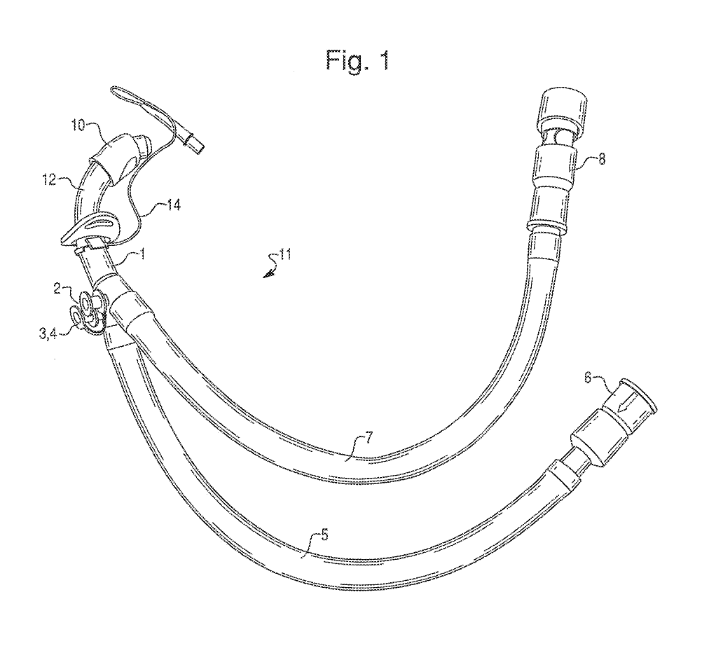

A first exemplary embodiment of the present invention is a positive pressure endotracheal device generally designated by reference numeral 11 in FIG. 1. The positive pressure endotracheal device 11 includes: a WYE molded endotracheal connector interface 1 suitably sized for the patient to be treated (i.e., adult, child, infant, etc.); a primary pressure release port 2 (preferably associated with the WYE connector 1; a pressure monitor port 3 (preferably associated with the WYE connector 1; a dispenser port 4 (preferably associated with the WYE connector 1; an inspiratory line/tubing 5 (preferably smooth walled) as a first limb of the WYE connector 1; a one-way valve 6 only connected to the inspiratory line/tube 5; an expiratory line 7 (preferably smooth walled) as a second limb of the WYE connector 1; and a positive end expiratory pressure (PEEP) valve 8 only connected to the expiratory line 7. A tracheal or endotracheal tube/line 12 is also shown with a deflated cuff 10 and associated inflator line 14. During use, the tracheal or endotracheal tube/line 12 is placed in the patient.

The positive pressure provides an exemplary method associated with the endotracheal/tracheal device 11 which includes the steps of orienting the endotracheal/tracheal WYE connector 1 so as to facilitate interface with the endotracheal tube/interface 12, placing the pressure release port 2 on the proximal end of the molded endotracheal tube/interface 12, positioning the pressure monitor port 3, vertically, on the molded endotracheal connector; establishing the dispenser port 4 whereby the same port may be used as either the pressure monitor port 3 or as the dispenser port 4, placing the internal one-way valve 6 inside/outside the inspiratory line 5 of the assembly so as to prevent sourced air from passing backwards through the inspiratory line 5, connecting the expiratory line (smooth walled) tubing 7 to the WYE connector 1, and placing the PEEP valve 8 distal end of the expiratory line 7 or otherwise within the flow path of the expiratory line 7.

In general the device 11 operates as follows. Air flows from a suitable source and passes through tubing into the one-way valve 6 which will not allow air to return into the inspiratory line 5 once air has passed through to the patient. The air flows through the molded endotracheal WYE interface 1 into the trachea thereby inflating the lungs. A cuffed 10 endotracheal tube 12 traps the air inside the lungs and prevents exhaled air passing around the cuff 10 and out through the patient nose or mouth. As the lung inflates, exhalation (passive) occurs forcing the exhaled lung volume out through the exhalation line 7. As the air follows the path of least resistance the PEEP valve 8 limits the amount of exhaled gas while allowing exhalation to occur.

In more detailed operation, the positive pressure endotracheal/tracheal device 11 works as follows. The source flow/O2 tank/wall O2/provides intermittent/continuous positive pressure. As flow enters the inspiratory line 5, a pressure differential is created as the flow enters the one-way valve 6. The one-way valve 6 will remain open as flow remains constant, and the source air pressure exceeds the pressure within the inspiratory line 5. The one-way valve 6 also will not allow exhaled exhaust to return through the inspiratory line 5. The inflation of the lungs occurs, producing an increased pressure gradient. Elastic recoil, exhalation, occurs due to an increased pressure gradient, and exhaled lung exhaust follows the path of least resistance through the expiratory line 7. The volume of the exhaled lung exhaust is limited by the use of a resistor, specifically (PEEP) positive end expiratory pressure valve 8. The resistor (PEEP valve 8) will limit the exhaled lung volume to a predetermined volume allowing exhalation to a more physiological way of breathing.

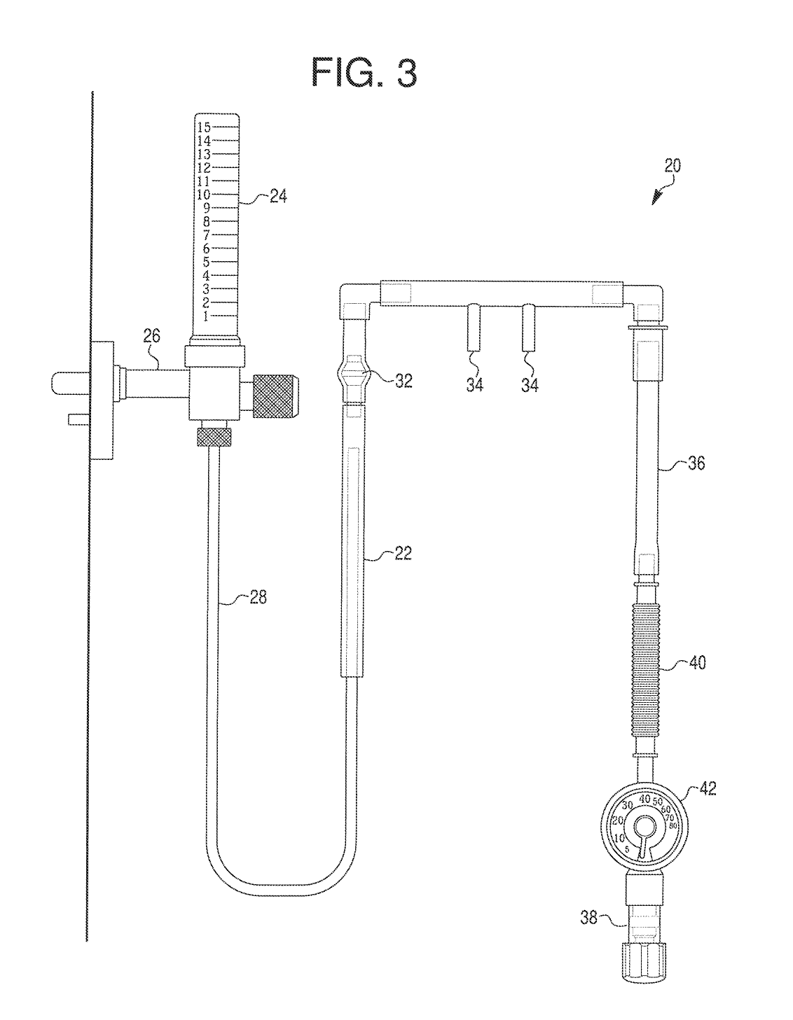

Referring now more particularly to FIG. 3, a positive pressure device according to another exemplary embodiment of the invention is generally designated by reference numeral 20. The positive pressure device 20 includes an inspiratory line 22, such as a tube, adapted to accept inspiratory air from a flow meter 24 representing a positive pressure air/oxygen source. In the embodiment illustrated in FIG. 3, the flow meter 24 is equipped with a pressure regulator 26. A line, such as a plastic tube 28, connects the flow meter 24 to the inspiratory line 22. By way of example, the plastic line 28 may be a tube twenty-five feet in length. It should be understood that the flow meter 24 and the plastic line 28 are optional, and do not necessarily form part of the positive pressure device 20. The flow meter 24 and the line 28 may comprise part of an overall system for use with the positive pressure device 20.

The positive pressure device 20 includes a one-way valve (e.g., micro check valve) 32 associated with the inspiratory line 22. The one-way valve 32 may be placed inside or outside the inspiratory line 22, and prevents sourced air from passing backwards through the inspiratory line 22. The one-way valve 32 may be, for example, 0.175 mm by 9.5 mm, and may be made of polypropylene, nylon, PVDF, polycarbonate, or other materials suitable for use as O-rings, e.g., silicon, Buna.

The positive pressure device 20 further includes a patient nasal interface 34 connected to a proximal end of the inspiratory line 22. The patient nasal interface 34 is suitably sized for the patient to be treated, for example, an adult, child, or infant. For example, the patient nasal interface 34 may include first and second nasal prongs for receipt in respective nostrils of the patient's nose. The patient nasal interface 34 is configured to receive the inspiratory air from the inspiratory line 22 and to conduct the inspiratory air into the nasal passage and thereby into the lungs of the patient. The patient nasal interface 34 is also connected to an expiratory line 36 configured to conduct air exhaled from the lungs of the patient through the nose at an exhalation pressure exceeding the positive air pressure. The expiratory line 36 may be smooth walls.

An adjustable positive end expiratory pressure (or PEEP) valve 38 is connected to the expiratory line 36 for restricting flow of the exhaled air while permitting release of the exhaled air from the expiratory line 36 through the PEEP valve 38 into the atmosphere at atmospheric pressure (760 mm Hg). The adjustable positive end expiratory pressure valve 38 is configured to cooperate with the one-way valve 32 to permit adjustment of an inflation pressure of the lungs of the patient. For example, the PEEP valve 38 may be an adjustable spring plastic clear body PEEP valve resistor (5-20 cm H₂O).

Optionally, an extendable/retractable line or tube 40 associated with a pressure manometer 42 connects the expiratory line 36 to the PEEP valve 38. The extendable/retractable line/tube 40 may be used as dead space ventilation to extend the exhalation time, allowing carbon dioxide to be manipulated. The respiratory system provides for the elimination of CO2 into the atmosphere. A longer e-time (exhalation time) will produce more CO2 within the exhalation line and the lungs. An increased level of CO2 induces an increase in ventilation. Alternatively hyperventilation involves over breathing, or a reduction in CO2. Increasing dead space (ventilation without perfusion) will increase CO2. The changes in CO2 levels may be recorded by blood samples and CO2 monitors. During a single breathing cycle, inspiratory time plus expiratory time equals total cycle time.

The pressure manometer 42 measures lung exhaust exhaled by the patient, typically in cmH2O. The PEEP valve continuously allows the lung exhaust to be eliminated to atmospheric pressure while restricting the lung exhaust flow. A continuous positive air pressure (CPAP) is created in the lungs that exceeds the positive air pressure provided by the positive pressure air source through the pressure regulator 26.

By way of example, the PEEP valve 38 may operate by adjusting the mount of lung exhaust in increments of 5 cm at a time. A manometer 42 measures lung exhaust in cmH2O (pressure) and the PEEP valve 38 continuously allows the lung exhaust to be eliminated to atmospheric pressures through adjustable and controlled release of the lung exhaust. The PEEP valve 42 restricts lung exhaust and hence lung pressure by adjusting the amount of exhaled lung exhaust in increments of, for example, 5 cm at a time by dialing the amount of restriction.

In an exemplary embodiment, the one-way valve 32 and the adjustable positive end expiratory pressure valve 38 are configured to continuously transmit maintain the positive air pressure from the positive pressure air source into the lungs of the patient when the patient is breathing spontaneously. The positive end expiratory pressure valve 38 is configured to provide controlled passive exhalation to atmospheric pressure while maintaining a sustained positive end expiratory pressure. By controlling the release of air through the PEEP valve 38 and preventing the backflow of air through the one-way valve, distension of the lungs occurs, and the lungs remain open to allow for oxygenation. The embodiment thus provides CPAP and positive end expiratory pressure (PEEP) simultaneously.

The device 20 operates as follows according to an exemplary embodiment of the invention. Air flows from a suitable source such as the flow meter 24. The flow meter 24 transmits pressured air from a positive pressure air source. The air source may include or consist of an oxygen source, such as an oxygen tank or wall source to provide intermittent and/or continuous positive pressure. The flow meter 24 measures the volume of air passing there though as, for example, volume per unit of time, such as liters per minute. The pressure regulator 26 cuts off or controls the flow of air at a predetermined pressure. For example, the pressure regulator 26 may be designed to lower the input pressure from, for example, 50 psi (pounds per square inch) down to a lower output pressure. For every liter per minute (1 pm) that the flow is increased, the oxygen concentration of air increases by four percent. For example, the flow meter may operate as follows: 1 lpm=24%; 2 lpm=28%; 3 lpm=32%; 4 lpm=36%; 5 lpm=40%; 6 lpm=44%; 7 lpm=48%; 8 lpm=52%; 9 lpm=56%; 10 lpm=60%; 11 lpm=64%; 12 lpm=68%; 13 lpm=72%; 14 lpm=76%; and 15 lpm=80%. Accordingly, as used herein, “air” encompasses air modified with increased oxygen content.

The air passes through inspiratory line 22 into the one-way valve 32. The one-way valve 32 remains open as flow passes through the valve 32. The one-way valve 32 does not allow exhaled exhaust to return through the inspiratory line 22. A pressure differential is created as the flow enters the one-way valve 32.

The air flows through the patient nasal interface 34 into the patient's airway and into the lungs, thereby inflating the lungs. As the lung inflates, exhalation (passive) occurs forcing the exhaled lung volume out through the exhalation line 36. As the air follows the path of least resistance through the expiratory line 36 towards the PEEP valve 38, the PEEP valve 38 limits the emission of exhaled gas from the system while allowing exhalation to occur. As insufflations occur the lungs expand creating an increasing pressure gradient. Exhaled lung exhaust follows the path of least resistance through the expiratory line 36. The volume of the exhaled lung exhaust is limited by the use of a resistor, specifically (PEEP) positive end expiratory pressure valve 38. The PEEP valve 38 limits the exhaled lung volume to a predetermined volume allowing exhalation to a more physiological way of breathing. By controlling the release of air through the PEEP valve 38, the pressure in the lungs, as measured by the pressure manometer 42, can be controlled.

The positive pressure device 20 may be operated to allow for the exchange of oxygen and carbon dioxide in the patient's lungs. The device 20 enables alveoli expansion continuously. Further, the device 20 may be operated automatically by adjustment of the PEEP valve without the opening and closing pressures associated with mechanical ventilation, and without the need for electric or battery operated devices. The device 20 is cost effective, safe, low maintenance, and physiologically consistent with normal breathing.

The above aspects and embodiments may be combined and practiced with one another in any combination. While specific versions of exemplary devices have been disclosed, it will be understood that reasonable variations, such as nasal, tracheal, mask applications fall within the scope of the invention and may be implemented by those of ordinary skill in the field having reference to this application.

What is claimed is:

1. A positive pressure nasal device, comprising:
an inspiratory line adapted to accept inspiratory air from a positive pressure air source at a positive air pressure;
a one-way valve connected to the inspiratory line to permit one-way flow of the inspiratory air through the inspiratory line;
a patient nasal interface connected to the inspiratory line and configured to receive the inspiratory air from the inspiratory line and conduct the inspiratory air into the lungs of the patient;
an expiratory line connected to the patient nasal interface and configured to conduct air exhaled from the lungs of the patient; and
an adjustable positive end expiratory pressure valve connected to the expiratory line for restricting release of the exhaled air from the expiratory line to atmospheric pressure, the adjustable positive end expiratory pressure valve being configured to cooperate with the one-way valve to permit adjustment of an inflation pressure of the lungs of the patient to reach a continuous positive air pressure that exceeds the positive air pressure provided by the positive pressure air source.

2. The positive pressure nasal device of claim 1, wherein:
the one-way valve and the adjustable positive end expiratory pressure valve are configured to continuously transmit and maintain the positive air pressure from the positive pressure air source into the lungs of the patient when the patient is breathing spontaneously, and
the positive end expiratory pressure valve is configured to provide passive exhalation to atmospheric pressure while maintaining a sustained positive end expiratory pressure.

3. The positive pressure nasal device of claim 1, wherein the patient nasal interface comprises nose prongs.

4. The positive pressure nasal device of claim 1, wherein the inspiratory line is connectable directly or indirectly to a flow meter comprising a pressure regulator.

5. The positive pressure nasal device of claim 1, further comprising plastic tubing connected to a distal end of the inspiratory line, the plastic tubing being directly connectable to a flow meter comprising a pressure regulator.

6. The positive pressure nasal device of claim 1, further comprising extendable retractable tubing connecting the expiratory line to the adjustable positive end expiratory pressure valve.

7. The positive pressure nasal device of claim 1 further comprising a pressure manometer and extendable retractable tubing connecting the expiratory line to the adjustable positive end expiratory pressure valve.

8. A method of using a positive pressure device, comprising:
delivering inspiratory air from a positive pressure air source through an inspiratory line to a patient wearing a patient nasal interface, the inspiratory line being associated with a one-way valve configured to permit one-way flow of the inspiratory air through the inspiratory line to the patient nasal interface; and
controlling an adjustable positive end expiratory pressure valve connected to the patient nasal interface through an expiratory line to adjust an inflation pressure of the lungs of the patient to reach a continuous positive air pressure that exceeds the positive air pressure provided by the positive pressure air source.

9. The method of claim 8, further comprising:
continuously transmitting and maintaining the positive air pressure from the positive pressure air source into the lungs of the patient when the patient is breathing spontaneously, and
providing passive exhalation to atmospheric pressure while maintaining a sustained positive end expiratory pressure.

10. The method of claim 8, wherein the patient nasal interface comprises nose prongs.

11. The method of claim 8, wherein the inspiratory line is connectable directly or indirectly to a flow meter comprising a pressure regulator.

12. The method of claim 8, further comprising plastic tubing connected to a distal end of the inspiratory line, the plastic tubing being directly connectable to a flow meter comprising a pressure regulator.

13. The method of claim 8, further comprising connecting the expiratory line to the adjustable positive end expiratory pressure valve with an extendable-retractable tubing.

14. The method of claim 8, further comprising measuring pressure with a pressure manometer.

15. A positive pressure tracheal device, comprising:
an inspiratory line adapted to accept inspiratory air from a positive pressure air source at a positive air pressure air;
a one-way valve connected to a distal end of the inspiratory line, the one-way valve configured to create an increased pressure gradient within lungs of a patient when the lungs are inflated and to allow the inspiratory air to flow into the inspiratory line in a single flow direction through the one-way valve, the one-way valve being connected only to the inspiratory line; and
a wye connector connected to a proximal end of the inspiratory line and configured to receive the positive pressure air from the inspiratory line and conduct the positive pressure air air to a cuffed endotracheal or tracheal tube adapted to introduce the positive pressure air through a patient's airway and into the patient's lungs; and
an expiratory line connected to the wye connector and configured to conduct air exhaled from the patient's lungs at an exhalation pressure exceeding the positive air pressure of the inspiratory air from the patient's lungs through the wye connector;
wherein the expiratory line includes an adjustable positive end expiratory pressure valve connected only to the expiratory line for restricting flow of the exhaled air while releasing the exhaled air from the expiratory line to atmospheric pressure, the adjustable positive end expiratory pressure valve being configured to adjust lung inflation pressure of the patient,
wherein the one-way valve and the adjustable positive end expiratory pressure valve are configured to continuously transmit and maintain the positive air pressure from the positive pressure air source into the lungs of the patient when the patient is breathing spontaneously, and
wherein the positive end expiratory pressure valve is configured to provide passive exhalation to atmospheric pressure while maintaining a sustained positive end expiratory pressure.

16. The device as in claim 15, further comprising:
a pressure release port on the wye connector.

17. The device as in claim 15, further comprising:
a pressure monitor port on the wye connector.

18. The device as in claim 15, further comprising:
a dispenser port on the wye connector.
